(19) United States
(12) Patent Application Publication
(10) Pub. No.: US 2016/0193436 A1
(43) Pub. Date: Jul. 7, 2016

(54) RESPIRATORY THERAPY APPARATUS, SENSORS AND METHODS

(71) Applicant: SMITHS MEDICAL INTERNATIONAL LIMITED, Ashford, Kent TN25 4BF (GB)

(72) Inventor: Mohammad Qassim Mohammad Khasawneh, Luton (GB)

(73) Assignee: SMITHS MEDICAL INTERNATIONAL LIMITED, Ashford, Kent TN25 4BF (GB)

(21) Appl. No.: 14/905,079

(22) PCT Filed: Jun. 10, 2014
(86) PCT No.: PCT/GB2014/000220

§ 371 Date: Jan. 14, 2016

(30) Foreign Application Priority Data
Jul. 19, 2013 (GB) 1312934.1

(51) Int. Cl. A61M 16/00

(52) U.S. Cl. CPC A61M 16/0006; A61M 2205/502

(57) ABSTRACT

Respiratory therapy apparatus includes an expiratory vibratory therapy device (100) of the kind that produces an alternating resistance to expiration through the device. The apparatus also includes a sensor (30) that is strapped to the chest of the patient. The sensor (30) includes a three-axis accelerometer (36) responsive to vibration in the chest and a display (34) on which the frequency of chest vibration and the length of the therapy session are represented. The display (34) can be switched between an upright and inverted configuration so that it can be viewed by the patient looking down at the sensor (30).

This invention relates to respiratory therapy apparatus of the kind including a respiratory therapy device of the kind arranged to produce an alternating resistance to breathing through the device.

The invention is also concerned with methods of evaluating patient use of respiratory therapy apparatus.

For patients with respiratory system diseases, such as asthma, COPD, cystic fibrosis and the like, hyper-secretion of mucus is a prominent pathophysiological feature and is often accompanied by impaired mucus transport. This imbalance between mucus transport and secretion results in mucus being retained in the respiratory system. Positive expiratory pressure (PEP) apparatus, that is, apparatus that presents a resistance to expiration through the device, are now widely used to help treat patients suffering from a range of respiratory impairments. More recently, such apparatus that apply chest physiotherapy by providing an alternating resistance to flow have been found to be particularly effective. One example of such apparatus is sold under the trade mark Acapella (a registered trade mark of Smiths Medical) by Smiths Medical and is described in U.S. Pat. No. 6,581,598, U.S. Pat. No. 6,776,159, U.S. Pat. No. 7,059,324 and U.S. Pat. No. 7,699,054. Other vibratory respiratory therapy (V-PEP) apparatus is available, such as “Quake” manufactured by Thayer, “AeroPEP” manufactured by Monaghan, “TheraPEP” manufactured by Smiths Medical and “IPV Percussionator” manufactured by Percussionaire Corp. The generated vibratory positive pressures work on mechanically reducing the viscoelasticity of sputum by breaking down the bonds of mucus macromolecules which enhances mucociliary clearance. Alternative apparatus such as “CoughAssist” manufactured by Philips are also available. Respiratory therapy apparatus can instead provide an alternating resistance to flow during inhalation.

Although these devices can be very effective, users often neglect to use them correctly or do not use them regularly at the prescribed frequency. It is very difficult to maintain a record of use of the device, especially when the patient is using it at home. The clinician often does not know whether deterioration in a patient's condition is because he has failed to use the device as prescribed or whether other factors are the cause. The effectiveness of treatment by such V-PEP devices is also critically dependent on the frequency and amplitudes on the generated vibration. Although there have been proposals to monitor operation of such devices such proposals have not addressed how to measure actual vibration within the lung.

It is an object of the present invention to provide alternative respiratory therapy apparatus.

According to one aspect of the present invention there is provided respiratory therapy apparatus of the above-specified kind, characterised in that the apparatus includes a sensor arranged to be placed in vibratory communication with the patient's chest, and that the sensor is arranged to provide signals responsive to vibration in the chest during use of the device.

The sensor preferably includes an accelerometer such as a three-axis accelerometer. The sensor preferably includes a display on which information is presented. The display may be switchable between an upright configuration and an inverted configuration for viewing by the patient looking down at the sensor. The sensor preferably displays information as to one or both of the following: frequency of vibration in the chest and duration of use of the therapy device. The apparatus preferably includes a support for supporting the sensor on the patient's chest, which may include an elastic strap. The respiratory therapy device is preferably an expiratory therapy device.

According to another aspect of the present invention there is provided a sensor for use in respiratory therapy apparatus according to the above one aspect of the present invention.

According to a third aspect of the present invention there is provided a sensor arranged to be placed in vibratory communication with the patient's chest, wherein the sensor is arranged to provide signals responsive to vibration in the chest during respiratory therapy of the kind produced by breathing through a device that provides an alternating resistance to breathing.

The sensor preferably includes a display on which information about vibration in the chest is displayed. The display may be switchable between an upright configuration and an inverted configuration for viewing by the patient looking down at the sensor.

According to a fourth aspect of the present invention there is provided a method of monitoring respiratory therapy including the steps of placing a sensor in vibratory communication with the chest of a patient, and utilising the output from the sensor while the patient uses a vibratory respiratory therapy device.

The output of the sensor is preferably utilised by displaying on the sensor an indication of frequency of vibration in the chest and viewing the sensor to determine if the therapy is being carried out effectively.

Apparatus including a vibratory PEP device will now be described, by way of example, with reference to the accompanying drawings, in which:

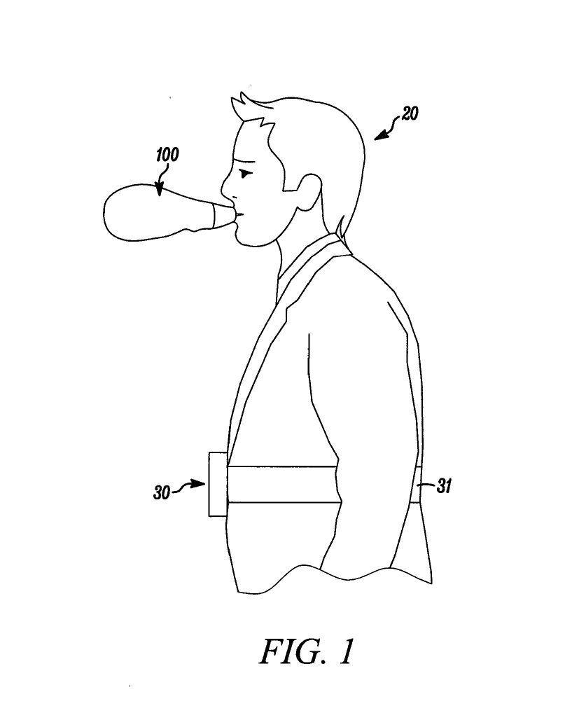

FIG. 1 illustrates the apparatus in use;

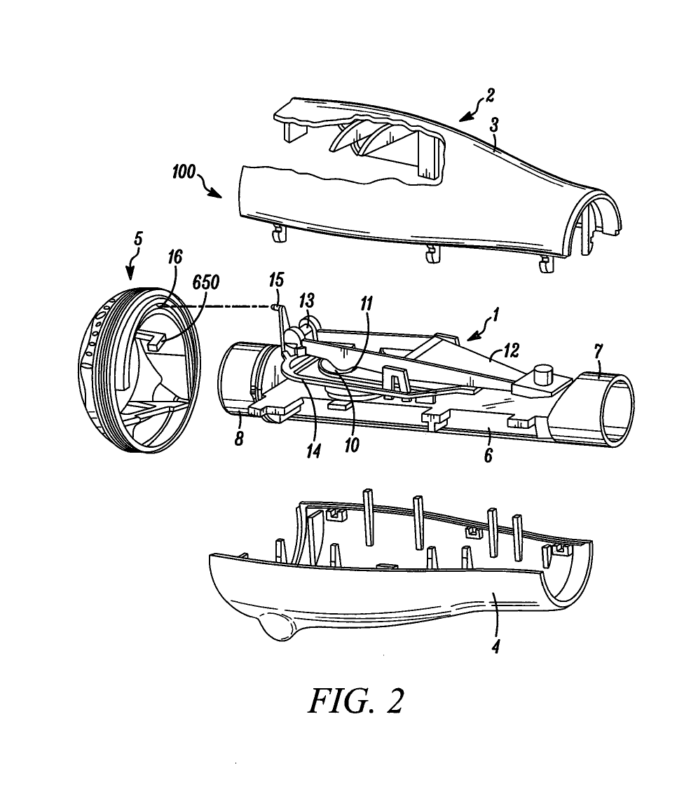

FIG. 2 is an exploded view of the respiratory therapy device;

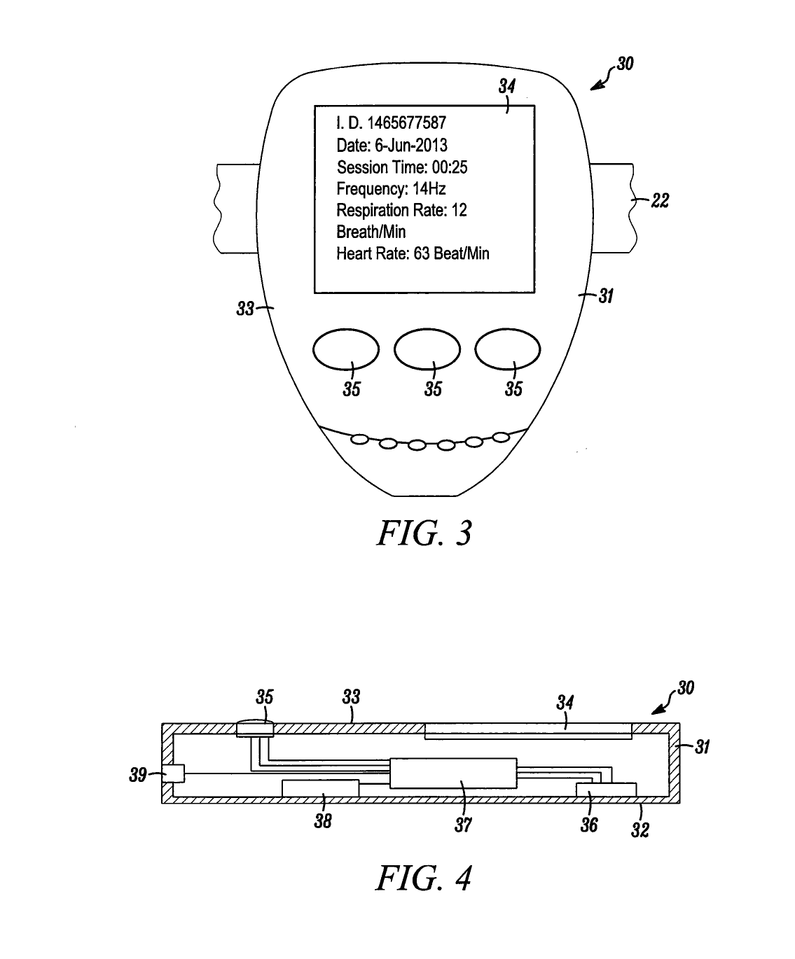

FIG. 3 is a front view of the sensor;

FIG. 4 is a schematic cross-sectional side elevation of the sensor;

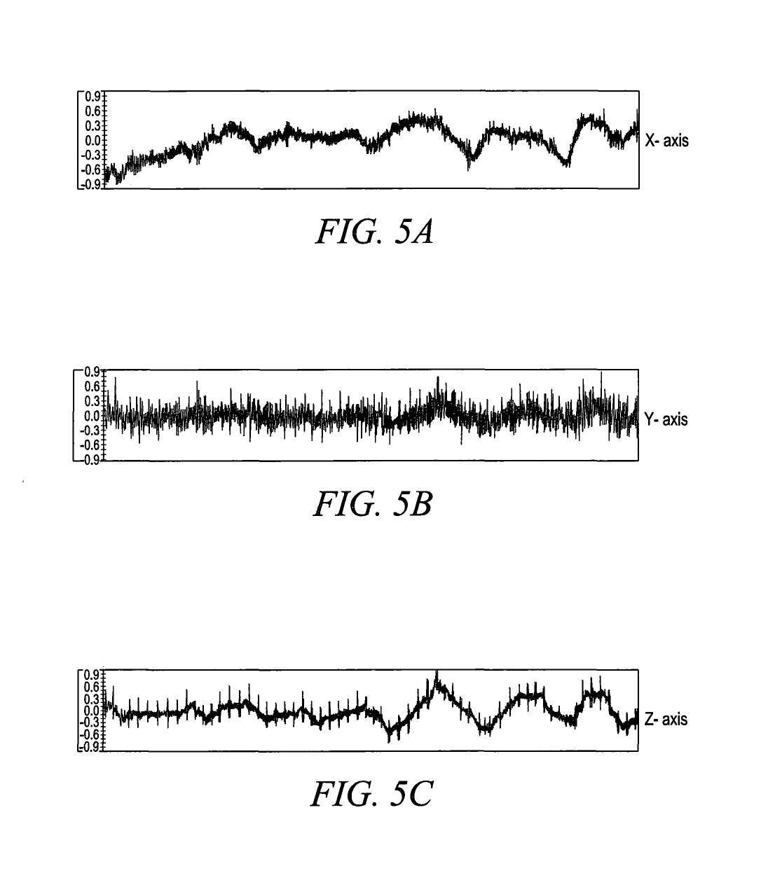

FIGS. 5A, 5B and 5C are graphs representing the outputs from an accelerometer in the sensor; and

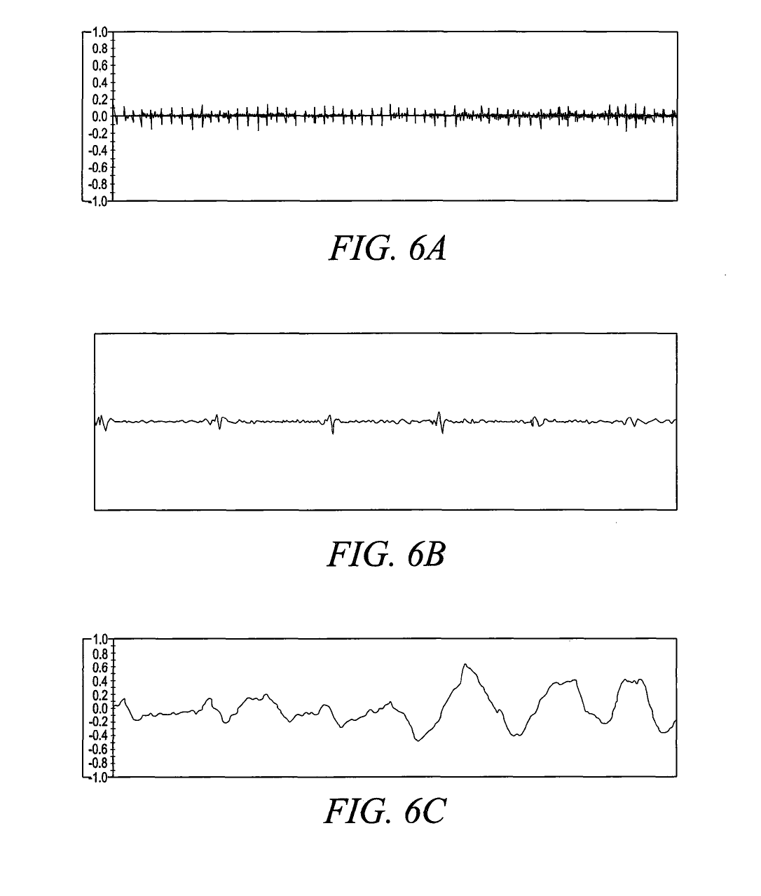

FIGS. 6A, 6B and 6C show signals derived after processing the accelerometer outputs.

With reference first to FIG. 1 there is shown a patient 20 breathing through a respiratory therapy device 100 and wearing sensor means 30 supported in contact with his chest by an elastic strap 22. The sensor means 30 is responsive to vibration in the lungs of the patient caused by use of the device 100 and is described in detail below.

The respiratory therapy device may be of any conventional kind that produces vibration within the user's lungs. The device 100 shown in FIG. 2 is an Acapella respiratory expiratory therapy device as sold by Smiths Medical. The device 100 comprises a rocker assembly 1 contained within an outer housing 2 provided by an upper part 3 and a lower part 4 of substantially semi-cylindrical shape. The device is completed by an adjustable dial 5 of circular section. The rocker assembly 1 includes an air flow tube 6 with a breathing inlet 7 at one end and an inspiratory inlet 8 at the opposite end including a one-way valve (not shown) that allows air to flow into the air flow tube 6 but prevents air flowing out through the inspiratory inlet. The air flow tube 6 has an outlet opening 10 with a non-linear profile that is opened and closed by a conical valve element 11 mounted on a rocker arm 12 pivoted midway along its length about a transverse axis. The air flow tube 6 and housing 2 provide a structure with which the rocker arm 12 is mounted. At its far end, remote from the breathing inlet 7, the rocker arm 12 carries an iron pin 13 that interacts with the magnetic field produced by a permanent magnet (not visible) mounted on an adjustable support frame 14. The magnet arrangement is such that, when the patient is not breathing through the device, the far end of the rocker arm 12 is held down such that its valve element 11 is also held down in sealing engagement with the outlet opening 10. A cam follower projection 15 at one end of the support frame 14 locates in a cam slot 16 in the dial 5 such that, by rotating the dial, the support frame 14, with its magnet, can be moved up or down to alter the strength of the magnetic field interacting with the iron pin 13. The dial 5 enables the frequency of operation and the resistance to flow of air through the device to be adjusted for maximum therapeutic benefit to the user.

When the patient inhales through the breathing inlet 7 air is drawn through the inspiratory inlet 8 and along the air flow tube 6 to the breathing inlet. When the patient exhales, the one-way valve in the inspiratory inlet 8 closes, preventing any air flowing out along this path. Instead, the expiratory pressure is applied to the underside of the valve element 11 on the rocker arm 12 causing it to be lifted up out of the opening 10 against the magnetic attraction, thereby allowing air to flow out to atmosphere. The opening 10 has a non-linear profile, which causes the effective discharge area to increase as the far end of the rocker arm 12 lifts, thereby allowing the arm to fall back down and close the opening. As long as the user keeps applying sufficient expiratory pressure, the rocker arm 12 will rise and fall repeatedly as the opening 10 is opened and closed, causing a vibratory, alternating or oscillating resistance to expiratory breath flow through the device. Further information about the construction and operation of the device can be found in U.S. Pat. No. 6,581,598, the contents of which are hereby incorporated into the present application.

With reference now to FIGS. 3 and 4, the sensor means 30 has an outer plastics housing 31 with a rear face 32 adapted to contact the skin on the chest and a front face 33 with a display screen 34 and three control buttons 35. Internally, the sensor 30 includes a conventional three-axis accelerometer 36 in contact with the internal surface of the rear face 32 so that vibration is transmitted to the accelerometer. The output of the accelerometer 36 is supplied to a processor 37 that is powered by a battery 38 and provides appropriate outputs to the screen 34. FIGS. 5A to 5B illustrate the three axes outputs from the accelerometer 36, that is, the x axis, y axis and z axis. These output signals are combined in the processor 37 and filtered to derive an output shown in FIG. 6A representative of the frequency. The processor 37 averages this output, converts it to a numerical value and displays this on the screen 34. The heart rate and respiratory rate can also be derived by the processor 37 from the accelerometer outputs as shown in FIGS. 6B and 6C respectively, which are also converted to a numerical value for display on the screen 34. The processor 37 additionally provides a representation on the screen 34 of the following: identification number of the user; date; and duration of the session. Preferably the sensor 30 also includes a communications port 39 by which data can be supplied to or from the processor 37, such as for reprocessing the processor or downloading information stored in a memory in the processor. This may be achieved by a cable connection or by wireless communication.

It will be appreciated that there are various ways in which the information derived by the sensor 30 can be presented to the patient and the clinician. The display information on the screen 34 may be in an upright alphanumeric configuration as shown, for viewing by the clinician, or the user may switch the display to an inverted, upside-down configuration by pressing one of the buttons 35 so that it is legible by the user on looking down. It is not essential that the information about frequency or respiration and heart rate be shown in numerical form. Instead it could be provided by displaying a legend on the display: “Frequency OK”, “Frequency Too High” or “Frequency Too Low”. Alternatively, the screen 34, or a part of the screen, could change colour to indicate whether frequency was too high or low, or a sound signal could be produced, especially for those with a visual impairment.

The display screen need not be built into the sensor itself but could be separate from the sensor and connected with it by a cable or by wireless communication. This would enable the user to position the display screen where it could be seen more easily.

The sensor need not include an integral three-axis accelerometer but could instead include one or more separate accelerometers or other devices responsive to vibration. The sensor could be held on the body in other ways apart from the elastic strap. If the patient were lying in a supine position it could just be rested on his chest. Alternatively, the sensor could be held on by some form of adhesive support or by an elastic vest or the like. It is not essential that the sensor be placed in direct contact with the chest but could be placed elsewhere provided that it was in vibratory communication with the lungs, that is, it could sense vibration within the lungs.

The invention is not confined to use with expiratory therapy devices but could be useful also in inspiratory vibratory therapy.

The present invention enables someone using an existing, conventional therapy device to be provided with useful data about its use. In particular, the user can be informed of the frequency and duration of the therapy, which have been found to be particularly important to ensure maximum effectiveness of the therapy. The invention enables the user to be made more aware of how well he is complying with the prescribed therapy programme so that he can modify his use of the device accordingly to achieve maximum benefit. The clinician is also able to check patient compliance so that he can identify whether any deterioration in a patient's condition is due to lack of compliance or if alternative treatment is needed.

1-15. (canceled)

16. Respiratory therapy apparatus including a respiratory therapy device of the kind arranged to produce an alternating resistance to breathing through the device, characterized in that the apparatus includes a sensor arranged to be placed in vibratory communication with the patient's chest, and that the sensor is arranged to provide signals responsive to vibration in the chest during use of the device

17. Respiratory therapy apparatus according to claim 16, characterized in that the sensor includes an accelerometer.

18. Respiratory therapy apparatus according to claim 17, characterized in that the accelerometer is a three-axis accelerometer.

19. Respiratory therapy apparatus according to claim 16, characterized in that the sensor includes a display on which information is presented.

20. Respiratory therapy apparatus according to claim 19, characterized in that the display is switchable between an upright configuration and an inverted configuration for viewing by the patient looking down at the sensor.

21. Respiratory therapy apparatus according to claim 19, characterized in that the sensor displays information as to one or both of the following: frequency of vibration in the chest and duration of use of the therapy device.

22. Respiratory therapy apparatus according to claim 16, characterized in that the apparatus includes a support for supporting the sensor on the patient's chest.

23. Respiratory therapy apparatus according to claim 22, characterized in that the support includes an elastic strap.

24. Respiratory therapy apparatus according to claim 16, characterized in that the respiratory therapy device is an expiratory therapy device.

25. A sensor for use in a respiratory therapy device to produce an alternating resistance to breathing through the device, the sensor arranged to be placed in vibratory communication with the patient's chest and to provide signals responsive to vibration in the chest during use of the respiratory therapy device.

26. A sensor arranged to be placed in vibratory communication with the patient's chest, wherein the sensor is arranged to provide signals responsive to vibration in the chest during respiratory therapy of the kind produced by breathing through a device that provides an alternating resistance to breathing.

27. A sensor according to claim 26, characterized in that the sensor includes a display on which information about vibration in the chest is displayed.

28. A sensor according to claim 27, characterized in that the display is switchable between an upright configuration and an inverted configuration for viewing by the patient looking down at the sensor.

29. A method of monitoring respiratory therapy including the steps of placing a sensor in vibratory communication with the chest of a patient, and utilising the output from the sensor while the patient uses a vibratory respiratory therapy device.

30. A method according to claim 29, characterized in that the output of the sensor is utilised by displaying on the sensor an indication of frequency of vibration in the chest and viewing the sensor to determine if the therapy is being carried out effectively.
